(19) United States
(12) Patent Application Publication
(10) Pub. No.: US 2022/0160441 A1
(43) Pub. Date: May 26, 2022

(54) SURGERY ASSISTANCE SYSTEM AND METHOD FOR GENERATING CONTROL SIGNALS FOR VOICE CONTROL OF MOTOR-CONTROLLED MOVABLE ROBOT KINEMATICS OF SUCH A SURGERY ASSISTANCE SYSTEM

(71) Applicant: Aktormed GmbH, Barbing (DE)

(72) Inventor: Robert Geiger, Metten (DE)

(21) Appl. No.: 17/452,813

(22) Filed: Oct. 29, 2021

(30) Foreign Application Priority Data
Nov. 25, 2020 (DE) 10 2020 131 232.6

(51) Int. Cl. A61B 34/30; G10L 15/22; G06T 7/00; G06T 7/90; A61B 1/00

(52) U.S. Cl. CPC A61B 34/30; G10L 15/22; G06T 7/0012; G06T 7/90; A61B 1/00149; A61B 1/3132; G06T 2207/10068; G06T 2207/20084; G06T 2207/30204; G06T 2207/30004; A61B 2034/302; G10L 2015/223

(57) ABSTRACT

The invention relates to a surgery assistance system for guiding an endoscope camera, at least a section of which can be introduced through a first surgical opening and is movable in a controlled manner in an operating space of a patient body. The system includes an endoscope camera for capturing images of the operating space and robot kinematics. The free end of the robot kinematics accommodates the endoscope camera by an auxiliary instrument carrier, the robot kinematics being movable by motor control for guiding the endoscope camera in the operating space and via control signals (SS) generated by a control unit, at least one voice control routine being executed in the control unit by which voice commands and/or voice command combinations in the form of voice data are captured, evaluated, and the control signals being generated on the basis thereof.

### BACKGROUND OF THE INVENTION

### 1. Field of the Invention

The invention relates to a surgery assistance system and a method for generating control signals for voice control of motor-controlled movable robot kinematics of a surgery assistance system.

### 2. Description of the Related Art

Surgery assistance systems, particularly those used for supporting medical interventions or operations, in particular minimally invasive operations, are sufficiently well known. They are used to improve safety, efficiency, and quality of outcomes in modern surgical operating rooms. Surgery assistance systems of such kind are especially important in the field of technology-driven, minimally invasive surgery.

Surgery assistance systems are frequently used to guide auxiliary surgical instruments, such as camera systems, in particular, “endoscope cameras.” For example, a surgery assistance system is known from German Patent No. DE 10 2007 019363 A1, via which for example an endoscope including a camera unit, that is to say an endoscope camera, is guided in a controlled manner. For this purpose, the surgery assistance system comprises robot kinematics designed to be drivable in a controlled manner by means of a plurality of drive units. With this robot kinematics, an endoscope camera mounted on an instrument holder, in particular the free camera end thereof, can be moved in a controlled manner in a three-dimensional space, in particular in an internal operating space in the context of a minimally invasive surgical procedure. For this purpose, the robot kinematics comprises for example at least one support column, at least two robot arms, and at least one instrument carrier accommodating the instrument holder.

Further, a method for guiding an endoscope camera by means of such a surgery assistance system depending on the manual actuation of at least one function key of an operating element, is known from German patent No. DE 10 2008 016 146 B4. Other assistance systems have also been disclosed which are configured to enable automated dynamic tracking of an endoscope camera based on the current position of a surgical instrument.

Control systems for such surgery assistance systems may also be equipped with a voice control system, as is known from German Patent No. DE 10 2017 101 782 A1 and PCT Published Patent Application No. WO 2013/186 794 A2. With such a voice control system, the surgeon can control the guidance of the auxiliary instrument with voice commands.

In particular, a surgical control system is known from German Patent No. DE 20 2013 012 276 U1 in which, besides a control using voice commands, the image data of the operating space acquired by an image acquisition apparatus can also be captured and subsequently evaluated with an image processing unit. However, with this solution as well, the guidance of the auxiliary instrument must still be controlled by the surgeon or a camera assistant on the instructions of the surgeon, who controls the auxiliary instrument depending on the image recordings and/or images displayed to them on a monitor unit. Disadvantageously, known voice controls systems are based on clearly defined command catalogs, which must be mastered by the surgeon or the camera assistant to ensure reliable control of the system. Intuitive operability by the surgeon or camera assistant is not possible with the known voice control systems.

Methods and apparatuses for recognizing image patterns or objects in images and recognizing a sequence of phonetic sounds or signs in voice signals are also known from the related art. In German Patent No. DE 100 06 725, for example, a method for processing speech using a neural network is described, which enables voice input by unformatted voice commands or spoken commands.

### SUMMARY OF THE INVENTION

It is an object of the invention to provide a surgery assistance system, particularly for medical interventions or operations, as well as an associated method for generating control signals for controlling the robot kinematics of such a surgery assistance system with voice commands, which system unburdens the surgeon to a large extent of the task of guiding the camera, and which is characterized particularly by intuitive operability and simplifies the process of camera guidance for the surgeon when performing minimally invasive surgical procedures.

An essential aspect of the surgery assistance system according to the invention resides in that at least one image evaluation routine is executed in the control unit and/or is provided therein, by which the image data captured are evaluated and classified continuously based on statistical and/or self-learning artificial intelligence methods. The object- and/or scene-related information regarding the surgical scene currently captured in the image by the endoscope camera are calculated by means of the continuous evaluation and classification of the image data, and the voice data detected are evaluated depending on the captured object- and/or scene-related information. Particularly advantageously, more intuitive voice control of the endoscope camera by the surgeon is made possible by the combined capture and evaluation of image and voice data relating to a surgical scene according to the invention, since the surgeon is able to use voice commands and/or voice command combinations which can be derived intuitively from the camera image to control the endoscope camera. For example, in order to exercise voice control, the surgeon may employ voice or language commands or voice command combinations that relate to individual objects appearing in the image and/or to their position and/or orientation in the image.

Also advantageously, the image analysis routine comprises a neural network with pattern and/or color detection algorithms for evaluating the captured image data. The pattern and/or color detection algorithms are preferably configured and trained to capture or detect objects or parts thereof present in the image, particularly surgical instruments or other medical tools or organs. The algorithms advantageously form part of a neural network that has been trained through the processing of a large number of training datasets.

In a preferred embodiment, the voice control routine is configured to evaluate the voice data based on statistical and/or self-learning artificial intelligence methods. The voice control routine comprises for example a neural network with sound and/or syllable recognition algorithms for evaluating the voice data, wherein the sound and/or syllable recognition algorithms are designed to detect sounds, syllables, words, breaks between words, and/or combinations thereof contained in the voice data. Thus, it becomes possible to use previously undefined or unformatted voice commands and/or voice command combinations, and more particularly image- and/or scene-related voice commands and/or voice command combinations to control the endoscope camera.

Also advantageously, the voice control routine executed in the control unit is configured to detect and evaluate object- and/or scene-related voice commands depending on the object- and/or scene-related information. By taking into consideration the object- and/or scene-related information, new voice commands whose contents relate to the surgical scene illustrated in image B can be processed automatedly to enable the generation of control signals. This has the effect of substantially increasing the scope of the control commands usable by the surgeon for actuation, which results in a more intuitive voice control.

In an advantageous further development of the invention, at least one assigned control signal is generated based on the captured object- and/or scene-related voice commands, via which at least the movement of the endoscope camera is controlled in respect of its direction, speed, and/or magnitude. Moreover, the voice control routine for capturing and evaluating object- and/or scene-related information may also be designed to capture and evaluate direction and/or speed information and/or associated magnitude information in the voice data. This in turn serves to enhance user-friendliness for the surgeon further.

Also advantageously, the endoscope camera may be designed to capture a two-dimensional or a three-dimensional image. Accordingly, three-dimensional endoscope cameras may also be used to capture the image data as well as conventional two-dimensional endoscope cameras. Advantageously, three-dimensional endoscope cameras of such kind may also be used to obtain depth information, which may be evaluated by the control unit as a further open- or closed-loop control parameter. The depth information obtained in this way may be used particularly advantageously for controlling and/or tracking guidance of the endoscope camera. For example, a predefined distance between the free end of the endoscope camera and a detected instrument tip may constitute an open- or closed-loop control parameter.

Particularly advantageously, a two-dimensional image coordinate system or a three-dimensional image coordinate system is assigned to the image via the image analysis routine. To ascertain the orientation or position of an object in the image, the coordinates of the object or of at least one marker or one marker point of the object are determined in the screen coordinate system. In this way, it is possible to determine the position of the detected object exactly in the screen coordinate system and from this to calculate control signals for guiding the endoscope camera in the spatial coordinate system.

In an advantageous design variant, surgical instruments and/or organs and/or other medical aids displayed as objects or parts of objects in the image are detected by the image analysis routine. In order to detect objects or parts of objects, one or more markers or marker points of an object can be detected particularly advantageously with the image analysis routine, wherein for example an instrument tip, a particular color or material property of the object, and/or an articulation point between a manipulator and the instrument shaft of a surgical instrument may serve as markers or marker points.

The detected markers or marker points are preferably evaluated using the image analysis routine in order to classify the surgical scene and/or the object(s) located therein, and on this basis, the object-related and/or scene-related information are identified. Then, the object-related and/or scene-related information identified by the image analysis routine is transferred to the voice control routine.

A further object of the invention is a method for generating control signals to actuate motor-controlled movable robot kinematics of a surgery assistance system for guiding the endoscope camera, in which the endoscope camera is arranged on the free end of the robot kinematics using an auxiliary instrument carrier, wherein at least a section of the endoscope camera can be introduced into the operating space of a patient body through a first surgical opening and at least one voice control routine is configured in a control unit in order to generate the control signals. Voice commands and/or voice command combinations are advantageously captured in the form of voice data and evaluated using the voice control routine, and on this basis, the control signals are generated. In the control unit at least one image capture routine is executed for continuous acquisition of the image data supplied by the endoscope camera relating to the operating space. According to the invention, the captured image data are evaluated and classified continuously based on statistical and/or artificial intelligence self-learning methods using an image analysis routine incorporated in the control unit. Object- and/or scene-related information regarding the surgical scene currently captured in the image by the endoscope camera is ascertained through the continuous evaluation and classification of the image data, wherein the captured voice data is evaluated depending on the captured object- and/or scene-related information. The method according to the invention thus facilitates more intuitive voice control of the endoscope camera derived from the current camera image to the advantage of the surgeon.

The captured image data are particularly advantageously evaluated in the image analysis routine using pattern and/or color detection algorithms of a neural network, wherein the objects or parts of objects displayed in the image, particularly surgical instruments or other medical tools, or organs, are detected using the pattern and/or color detection algorithms. The pattern and/or color detection algorithms are part of a “trained” neural network, which enables a reliable evaluation of the image data in real-time.

In an advantageous variant of the method according to the invention, in order to detect the objects or object parts one or more markets or marker points of an object are detected with the image analysis routine, wherein for example an instrument tip, particular color, or material properties of the object and/or an articulation point between a manipulator and the instrument shaft of a surgical instrument serve as markers or marker points. The markers or marker points detected are advantageously evaluated using the image analysis routine in order to classify the surgical scene and/or the objects located therein, and the object-related and/or scene-related information is determined on this basis.

Also advantageously, the voice data captured in the voice control routine are evaluated by sound and/or syllable recognition algorithms of a neural network. Preferably, sounds, syllables, words, breaks between words, and/or combinations thereof contained in the voice data are captured by the sound and/or syllable recognition algorithms. The object-related and/or scene-related information determined by the image analysis routine is transferred to the voice control routine. In the voice control routine, object- and/or scene-related voice commands are acquired and are evaluated depending on the transferred object-related and/or scene-related information.

In a further advantageous variant, a two-dimensional image coordinate system is assigned to the image by the image analysis routine, and the coordinates of the object or at least one marker or marker point of the object are calculated in the screen coordinate system in order to determine the orientation or position of an object in the image. In this way, it is possible to obtain a reliable position determination and generate associated control signals derived therefrom.

Within the meaning of the invention, the terms “approximately”, “substantially” or “roughly” are understood to mean deviations of +/−10%, preferably +/−5% from the respective exact value, and/or deviations in the form of changes that are insignificant for the function.

Further developments, advantages, and application capabilities of the invention are also described in the following description of exemplary embodiments and the figures. In this context, all features which are described and/or illustrated either individually or in any combination constitute an object of the invention, regardless of whether they are summarized or referenced in the claims.

### BRIEF DESCRIPTION OF THE DRAWINGS

FIG. 1 shows an exemplary perspective side view a surgery assistance system.

FIG. 2 shows an exemplary schematic sectional view through a patient body with an endoscope camera, at least a portion of which is accommodated in the operating space, and a medical surgery instrument.

FIG. 3 shows an exemplary image of the operating space generated by the endoscope camera.

FIG. 4 shows an exemplary schematic block diagram of the control unit and the units connected to it, and the control and evaluation routines executed therein.

FIG. 5 shows an exemplary flowchart of a variant of the image evaluation routine according to the invention.

FIG. 6 shows an exemplary flowchart of a variant of the voice control routine according to the invention.

### DETAILED DESCRIPTION OF THE INVENTION

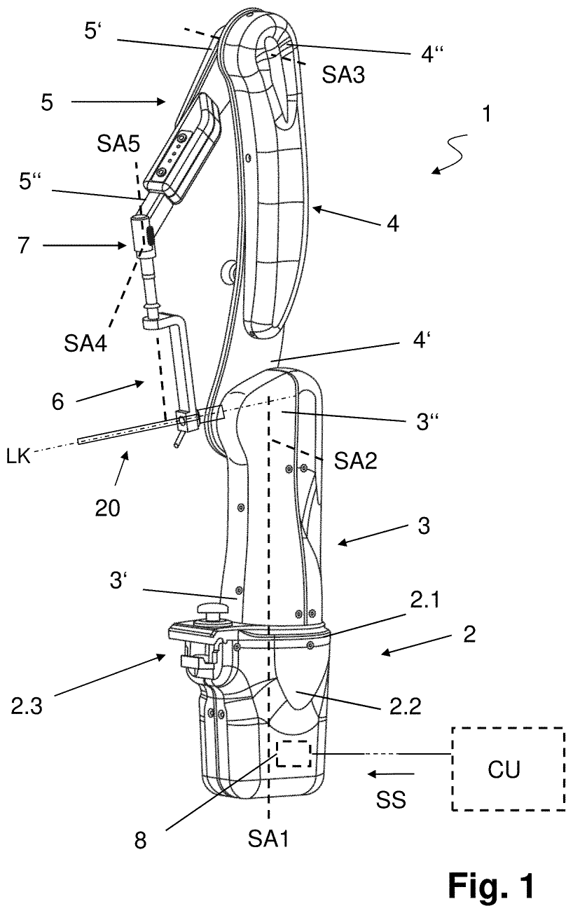

FIG. 1 shows an example of a surgery assistance system 1 for guiding an endoscope camera 20 in a patient body 10 during medical procedures or operations. The endoscope camera 20 consists essentially of an endoscope 21 and a camera unit 22 arranged at the free end thereof, the camera unit preferably being located outside the patient body 10.

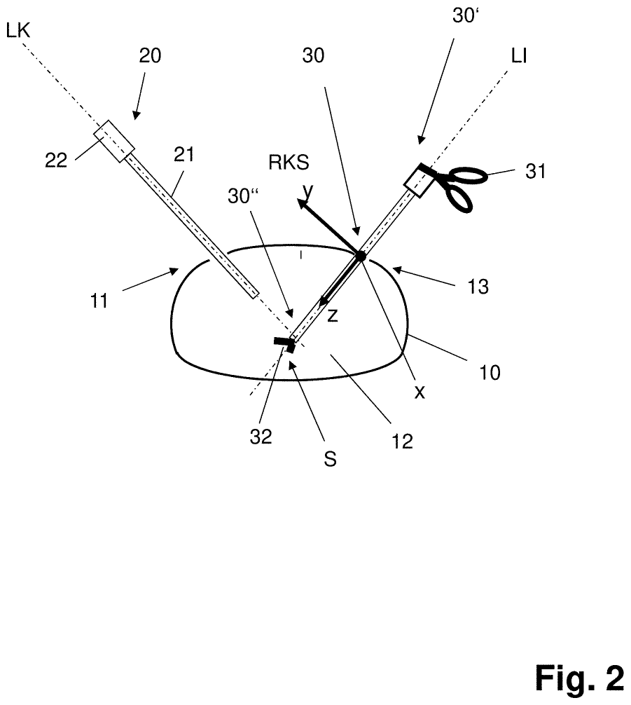

Such endoscope cameras 20 are preferably used in minimally invasive operations. For this purpose, they are introduced into an operating space 12 in the patient body 10 through a small first surgical opening 11. The actual surgical instrument 30 for performing the medical procedure is introduced into the operating space 12 through a second surgical opening 13 in the patient body 10. The first and second surgical openings 11, 13 are frequently referred to as “trocar” or “trocar points” in the literature. FIG. 2 is a schematic representation of an example of a typical operating situation with a section through a patient body 10 in the minimally invasive operating region.

Using the endoscope camera 20 and its camera unit 22, respectively, image recordings and/or images B of the operating space 12, including of the tip S of the surgical instrument 30 in the operating space 12, are then generated continuously in the form of image data BD, and these are then displayed to the surgeon during the medical procedure or the minimally invasive operation on a monitor unit, which is not shown in the figures.

The surgeon can monitor the progress of the operation with the aid of the current pictures B (“live images”) from the operating space 12 which is displayed on the monitor unit and guide the surgical instrument 30 visible in image B accordingly. In order to be able to see a current and optimal image B of the operating space 12, particularly the surgical instrument 30 at all times, the surgeon needs to be able to actuate and dynamically guide the endoscope camera 20 as intuitively as possible, not least in order to minimize the burden on the surgeon presented by the task of guiding the camera during the surgical procedure. In this regard, the optimal display of the “field of interest” is particularly important. In this context, it is particularly desirable if the tip S of the surgical instrument 30 is located in the center or middle of image B which is displayed to the surgeon.

The surgery assistance system 1 according to the invention makes it possible for the surgeon to operate and guide the endoscope camera 20, intuitively and in a user-friendly manner using context-dependent “voice control”. A surgery assistance system 1 which is controllable in such a way consists for example of a base unit 2 and robot kinematics comprising a system of multiple arms, in particular robot arms, wherein in the present embodiment the robot kinematics includes a support column 3, a first and a second robot arm 4, 5 and an auxiliary instrument carrier 6. The auxiliary instrument carrier 6 is attached to the second robot arm 5, by a hinged joint, for example, specifically here by an angled joint part 7. The auxiliary instrument carrier 6 is designed to accommodate the endoscope camera 20, directly for example. The described construction of the robot kinematics of the surgery assistance system 1 is shown for exemplary purposes in the perspective view of the surgery assistance system 1 of FIG. 1.

The base unit 2 further comprises for example a carrier plate 2.1, a preferably multi-part base housing 2.2, and at least one fastening element 2.3, by means of which the preferably portably configured surgery assistance system 1 or the base unit 2 can be fastened for example to the side of an operating table (not shown in the figures). The base housing 2.2 houses at least a control device 8 and, optionally, further functional units, which may or may not cooperate with a computer system (not shown).

The support column 3 includes an upper and a lower end section 3′, 3″. The base unit 2 of the surgery assistance system 1 is connected to the lower end section 3′ of the support column 3 of the robot kinematics to be pivotable in a controlled manner about a first pivot axis SA1. The first pivot axis SA1 extends vertically to the installation plane of the surgery assistance system 1 or the operating plane or the plane of an operating table.

The first robot arm 4 further includes a first and a second end section 4′, 4″, wherein the first end section 4′ of the first robot arm 4 is connected to the upper end section 3″ of the support column 3 opposite the base unit 2 to be pivotable in a controlled manner about a second pivot axis SA2, and the second end section 4″ of the first robot arm 4 is connected to a first end section 5′ of the second robot arm 5 to be pivotable in a controlled manner about a third pivot axis SA3.

The second robot arm 5 includes a second end section 5″ opposite the first end section 5′, on which in the present embodiment the angled joint part 7 is provided to be rotatable about a fourth pivot axis SA4. The angled joint part 7 is constructed to accommodate a connecting portion of the auxiliary instrument carrier 6 in such a way that the carrier is detachable and also rotatable about a fifth pivot axis SA5. The opposite free end of the auxiliary instrument carrier 6 forms an instrument holder, which is preferably designed to hold an endoscope camera 20.

The first pivot axis SA1 extends perpendicularly to the installation plane or operating plane, and the second and third pivot axes SA2, SA3 extend parallel to each other, whereas the first pivot axis SA1 is aligned perpendicularly to the second or third pivot axis SA2, SA3.

Several drive units (not shown in the figures) are provided for driving the robot kinematics of the surgery assistance system 1, and these are designed to be actuatable via at least one control device 8, preferably independently of each other. The drive units are preferably integrated or accommodated in the base unit 2, the support column 3, and/or in robot arms 4 to 6. The drive units may be embodied for example as hydraulic drives or electrical drives, in particular linear motor units or spindle motor units.

At least one control device 8 is preferably accommodated in the base unit 2 of the surgery assistance system 1 and serves to generate control signals for actuating the drives or drive units for pivoting the motorized robot kinematics in a controlled manner about the predefined pivot axes SA1 to SA5 and/or for holding the robot kinematics in a predefined holding position in a Cartesian coordinate system.

The support column 3 extends vertically, substantially along the first pivot axis SA1, i.e., it is designed to be rotatable approximately about its own longitudinal axis. The first and second robot arms 4, 5 also extend substantially along a straight line, which preferably extends perpendicularly to the second and third pivot axes SA2, SA3 respectively. In the present embodiment, at least the first robot arm 4 is lightly curved.

In order to adjust the starting position of the surgery assistance system 1 and/or calibrate the control device 8 with reference to the first surgical opening 11 or trocar point, through which the endoscope camera 20 will be introduced into the operating space, a registering routine is provided, by which the surgery assistance system 1 is registered before the operation, for example, in that a registering scanner (not shown in the figures) is guided to the region of the patient, already in position on the operating table, in which the first surgical opening 11 for introducing the endoscope camera 20 is provided. Following this calibration, the surgery assistance system 1 is ready for use in guiding the endoscope camera 20.

FIG. 2 shows an exemplary schematic side view of an endoscope camera 20 introduced into the operating space 12 in a patient body 10 through the first surgical opening 11. FIG. 2 also shows a surgical instrument 30 introduced into the operating space 12 via the second surgical opening 13. The second surgical opening 13 here forms for example the origin of a Cartesian spatial coordinate system RKS having the spatial axes x, y, z.

In FIG. 2 the x-axis of the Cartesian spatial coordinate system RKS extends for example perpendicularly to the drawing plane, the y-axis of the Cartesian spatial coordinate system RKS extends perpendicularly to the longitudinal axis LI of the surgical instrument 30, while the z-axis extends along the longitudinal axis LI of the surgical instrument 30 or is coincident therewith. The origin is located in the region of the second surgical opening 13. With such an orientation of the Cartesian spatial coordinate system RKS, a rotation about the longitudinal axis LI of the surgical instrument 30 is advantageously equivalent to one rotation about the z-axis, which allows a simplified evaluation of a rotational movement about the longitudinal axis LI of the surgical instrument 30.

The surgical instrument 30 has for example at a free end 30′ at least one, preferably two grip elements 31, which are constructed for example in the form of two grip rings, each with an adjoining connecting shaft. A function element 32, for example, a gripping or cutting element arranged on the opposite free end 30″ of the medical instrument or surgical instrument 30, can be actuated via at least one of the grip elements 31. In this context, the function element 32 is the tip S of the surgical instrument 30, which is in the operating space 12 during the procedure and is captured by the endoscope camera 20. The free end 30′ is arranged outside the patient body 10 and forms the gripping region of the surgical instrument 30.

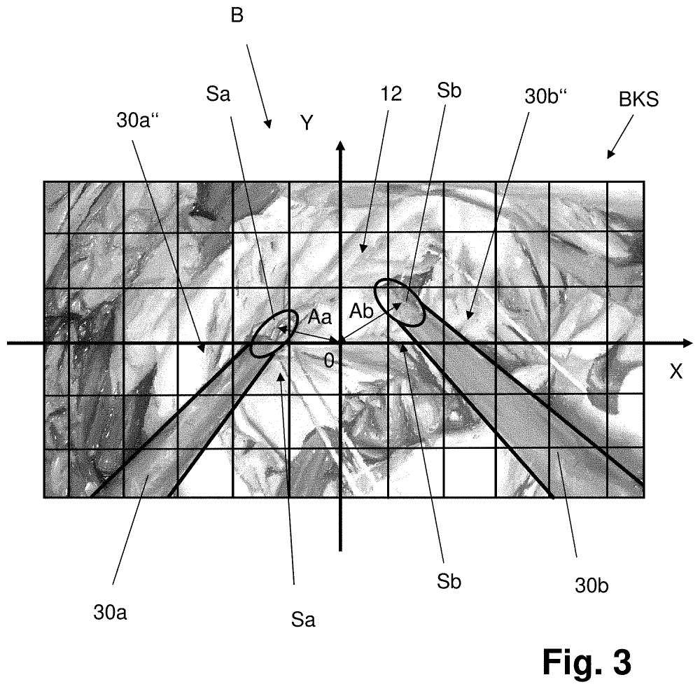

FIG. 3 shows an example of an image B of the operating space 12 which is captured in the form of image data BD by the endoscope camera 20 and displayed to the surgeon on a monitor unit in the form shown, for example. Image B shows an example of a first and a second surgical instrument 30a, 30b, and in the middle of image B the tips Sa, Sb of free ends 30a″, 30b″ of the medical operating instruments 30a, 30b, which are at least partially in contact with the organs shown. At least parts or sections of individual organs are also discernible in image B.

In order to control the movement of the endoscope camera 20, particularly to guide it dynamically to follow a surgical instrument 30, 30a, 30b in the operating space 12, the surgery assistance system 1 includes a control unit CU, by means of which control signals SS are generated relating preferably to the spatial coordinate system RKS. These are then transferred to the control device 8, by which a corresponding actuation of the drives or drive units of the robot kinematics is executed for controlled, motor-driven pivoting of the support column 3 and/or the robot arms 4, 5 of the robot kinematics to initiate a rotating and/or pivoting movement about the predefined pivot axes SA1 to SA5 and/or stopping of the robot kinematics in a predefined stopping position with reference to the spatial coordinate system RKS. The control device 8 includes a robot control routine, which generates the actuation signals required for actuating the various drive units of the robot kinematics. In particular, the robot control routine serves to calculate the respective target position to which the robot kinematics is to move depending on the control signals SS transmitted, starting from an existing actual position, relative in each case to the spatial coordinate system RKS, and generates the actuation signals needed therefor.

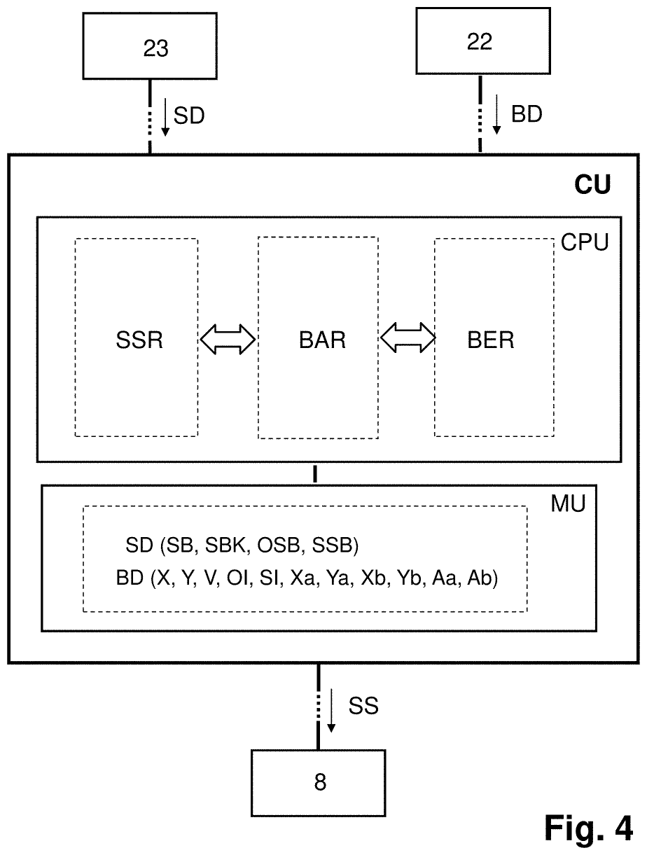

The control unit CU comprises for example at least one processor unit CPU and at least one memory unit MU. The processor unit CPU is preferably made up of at least one or more powerful microprocessor units. In the processor unit CPU of the control unit CU, at least one voice control routine SSR is executed which is designed to capture and evaluate the voice commands SB and/or voice command combinations SBK received in the form of voice data SD and generate control signals SS on the basis thereof. The control signals SS generated by the voice control routine SSR are then transmitted by the control unit CU to the control device 8 which is provided for actuating the robot kinematics 3, 4, 5. FIG. 4 shows an example of a schematic block diagram of a control unit CU according to the invention, which in the present embodiment is connected to the control device 8, the camera unit 22 of the endoscope camera 20, and a microphone unit 23.

The microphone unit 23 is provided for capturing voice commands SB and/or voice command combinations SBK. It may be integrated into the endoscope camera 20, for example, or otherwise assigned to the endoscope camera 20 or connected thereto. Alternatively, the microphone unit 23 may also be embodied as a mobile unit which may be fastened detachably in the area of the surgeon's head, for example. The microphone unit 23 may have the form of a wireless “headset”, for example, or also the form of a directional microphone arranged in the operating theatre. The voice commands SB and/or voice command combinations SBK captured by the microphone unit 23 is preferably transmitted via a wireless data link to the control unit CU where they are transferred via a suitable interface unit to the processor unit CPU for further processing.

The “voice control” implemented in the control unit CU enables the surgeon to control the guidance of the endoscope camera 20 or the dynamic tracking of the endoscope camera 20 to follow the surgical instrument 30, 30a, 30b by inputting voice commands SB and/or voice command combinations SBK. The use of a “voice control” to enable at least partial control or dynamic tracking of an endoscope camera 20 of a surgery assistance system 1 is known in principle, wherein this requires the input of predefined control commands SB and/or voice command combinations SBK, which preferably reflect control via an operating element with predefined movement directions. In the course of this process, it is also possible to adapt the acquisition parameters of the endoscope camera 20 to reflect changed procedure conditions by the input of predefined voice commands SB. However, a drawback associated with known systems is that intuitive voice control, i.e., adapted to the current surgical scene, is not possible. Indeed, voice input is still a complex undertaking for the surgeon, or input is made by a camera guidance assistant who receives corresponding instructions from the surgeon. This is where the invention starts and offers the surgeon more intuitive, and consequently more user-friendly voice control.

Besides the voice control routine SSR, according to the invention at least one image capture routine BER is provided in control unit CU and is designed to perform continuous acquisition of the image data BD supplied by the endoscope camera 20 regarding the operating space 12 and any medical surgical instruments 30, 30a, 30b located therein. The image capture routine BER is also executed in the processor unit CPU of the control unit CU, to which the image data BD supplied from the endoscope camera 20 are made available preferably continuously for further processing via a further interface unit.

According to the invention, an image analysis routine BAR which continuously evaluates and classifies the images B captured by endoscope camera 20 and the associated image data BD on the basis of statistical and/or artificial intelligence self-learning methods, particularly using a neural network, is assigned to the image capture routine BER in the control unit CU. The image data BD is preferably evaluated and classified by means of suitable pattern and/or color detection algorithms, which are preferably part of a neural network. The classification of the image data BD allows automated detection of predefined objects in image B captured by the endoscope camera 20, through which additional object- and/or scene-related information OI, SI about the surgical scene represented in image B can be ascertained. For example, through the image analysis routine BAR, which is also executed in the processor unit CPU, a surgical instrument 30, 30a, 30b which is visible in the currently captured image B from the endoscope camera 20 may be identified, and following this its position and/or orientation may be calculated in a two-dimensional Cartesian screen coordinate system BKS assigned to image B. For this purpose, the coordinates X, Y in the screen coordinate system BKS of at least one marker or marker point of the object, for example of the surgical instrument 30, 30a, 30b are determined. In addition to the coordinates X, Y of a marker or marker point of the object, the orientation of an object in image B can also be described using one or more vectors V.

Thus, a two-dimensional Cartesian screen coordinate system BKS is assigned to the image data BD captured by the endoscope camera 20, which can be represented as images B in a two-dimensional form on the monitor unit, by the image analysis routine BAR. For this purpose, the two-dimensional Cartesian screen coordinate system BKS has a first, horizontal image axis X and a second, vertical image axis Y, wherein the origin of the two-dimensional Cartesian screen coordinate systems BKS is preferably fixed at the center of the image displayed on the monitor unit, i.e., it is coincident with the middle of the image.

In this way, coordinates X, Y can be assigned to the pixels or pixel regions in the two-dimensional Cartesian screen coordinate system BKS that form image B and are represented on the monitor unit. This then makes it possible to divide the images B or the associated image data BD captured by the endoscope camera 20 into a predefined coordinate system BKS, with which the positions of individual objects, in particular the surgical instruments 30, 30a, 30b, can be determined by calculating the associated coordinates X, Y in the two-dimensional Cartesian screen coordinate system BKS. However, for the invention, objects may also be organs or parts thereof that are visible in image B, and other medical tools such as clamps, screws, or parts thereof.

In a variant of the invention, the endoscope camera 20 may also be designed for the capture of three-dimensional images B of the operating space, with which additional depth information is obtained. The image data BD delivered by a three-dimensional endoscope camera 20 of such kind are used for generating three-dimensional images B, which are displayed for example on a correspondingly configured monitor unit. It may be necessary to use 3D glasses to view the three-dimensional images B represented on the monitor unit. The design and operating principle of three-dimensional endoscope cameras 20 are known per se.

When a three-dimensional endoscope camera 20 is used, a three-dimensional Cartesian screen coordinate system is assigned to the image data BD, that is to say, a further coordinate axis, the Z-axis, is added to the two-dimensional Cartesian screen coordinate system BKS. The Z-axis is preferably coincident with the longitudinal axis of the endoscope 21. The position of a pixel in the three-dimensional image B is captured in the three-dimensional screen coordinate system by a specification of the associated X, Y, and Z coordinates.

In order to enable the detection and reliable differentiation of different objects, for example, different surgical instruments 30, 30a, 30b or organs or other medical tools such as surgical clamps, etc. in the captured image data BD, it is necessary to evaluate many real surgery scenarios beforehand. For this purpose, “training datasets” are generated in clinical studies when real medical, preferably minimally invasive operations are conducted. These training datasets comprise large quantities of image data BD from actual surgical procedures, each of which is annotated before they are used again, i.e., the various objects represented therein, such as the abovementioned surgical instruments 30, 30a, 30b are classified by kind or type, segmented with pixel-precise accuracy, and multiple markers or marker points are set which describe the structure and position of the surgical instruments 30, 30a, 30b. In order to differentiate between the different surgical instruments 30, 30a, 30b, in the context of the clinical studies the surgical instruments 30, 30a, 30b may be furnished with individual markers, color markers, or codes for example, which are applied to predefined instrument parts and/or sections. The markers applied to the respective instruments may be detected in the training datasets by the use of special pattern and/or color detection algorithms, and on this basis, the type, number, and/or position of the surgical instruments 30, 30a, 30b present in image B can be determined and classified.

From the great quantity of image data BD that is ascertained and annotated in clinical studies using surgical instruments 30, 30a, 30b bearing predefined markers, it is then possible to apply statistical and/or self-learning methods of artificial intelligence (“Deep Learning Methods”) particularly using deep neural networks to also determine further characteristic features of the surgical instrument 30, 30a, 30b of the example or other objects as well, which then form the “knowledge base” for a “self-learning” image analysis routine BAR. The “self-learning” image analysis routine BAR is “trained” through the evaluation of a large number of training datasets, and the objects that are detectable therewith, such as the surgical instruments 30, 30a, 30b, are classified accordingly. This then makes it possible to establish an automated recognition of objects in the camera image B based on the “trained” image analysis routine BAR without applying additional markers to the objects, by using the markers or marker points of the objects that can be derived in and of themselves from image B and the associated image data BD to evaluate or analyze the image data BD.

This approach may also pave the way for example to implement an at least partly automated tracking of the objects during the surgical procedure, in which both the movement history and the movement speed of an object detected in image B, such as a surgical instrument 30, 30a, 30b, is evaluated and used for at least partly automated control of the endoscope camera 20. In this context, the self-learning methods and/or algorithms utilized for this purpose then enable the processing not only of structured but also unstructured data such as the available image data BD, and in particular voice data SD as well.

A characteristic feature of a surgical instrument 30, 30a, 30b may be for example a uniquely identifiable point of a surgical instrument 30, 30a, 30b, such as the instrument tip S, Sa, Sb, or an articulation point between the manipulator and the instrument shaft, which is detectable using the “trained” image analysis routine BAR as a marker point of the surgical instrument 30, 30a, 30b in and of itself without the application of additional markers. Examples of different surgical instruments 30, 30a, 30b that can be recognized and classified correspondingly in the image analysis routine BAR include for example a forceps, scissors, a scalpel, etc. Characteristic features of organs or other medical aids may also serve as markers or marker points.

A marker or marker points may also be formed by the centroid of the surface section displayed in the image, for example, an organ or surgical instrument 30, 30a, 30b. Particularly in the case of elongated objects such as, for example, surgical instruments 30, 30a, 30b, apart from the instrument tip S, Sa, Sb a vector V extending or orientated along the longitudinal axis of the surgical instrument 30, 30a, 30b may also be captured as a further marker or marker points in image B, indicating the spatial orientation of the elongated object, particularly the surgical instrument 30, 30a, 30b, in image B or image coordinate system BKS.

One consideration of particular importance is that it must also be possible to differentiate reliably between several objects displayed in image B, for example, different surgical instruments 30, 30a, 30b and/or organs and/or other medical tools which are positioned partly on top of each other or overlapping in image B. In this case, for example, an instrument 30a in the capture area of the endoscope camera 20 may be located above or below another instrument 30b, or it may be partly obscured by an organ. Particularly when three-dimensional endoscope cameras 20 are used, it is possible to distinguish reliably between overlapping objects due to the additional depth information contained in the image data BD.

With due consideration for the stated boundary conditions, the images B that are captured in the form of image data BD by the endoscope camera 20 during a surgical procedure are continuously evaluated and classified with the “trained” image analysis routine BAR based on statistical and/or artificial intelligence self-learning methods, particularly with the aid of a neural network. According to the invention, it is possible to capture both two-dimensional and three-dimensional images B based on the image data BD. Through continuous evaluation and classification of the image data BD, object- and/or scene-related information OI, SI about the surgical scene currently in image B captured by the endoscope camera 20 are calculated, and voice commands SB and/or voice command combinations SBL that are captured by the voice control routine SSR, i.e. currently input, are evaluated in the form of voice data SD depending on the surgical scene captured, i.e. taking into account the determined object- and/or scene-related information OI, SI, i.e. context-dependent voice guidance or voice control for guiding the endoscope camera 20 is made available through the surgery assistance system 1 according to the invention.

The voice control routine SSR according to the invention is not only designed for evaluating predefined voice commands SB and/or voice command combinations SBK with predefined contents such as direction information or magnitude information, but also for evaluating voice data SD with object- and/or scene-related contents, hereinafter referred to as object- and/or scene-related voice commands OSB, SSB. For this purpose, the voice control routine SSR according to the invention is configured to carry out a continuous evaluation of the captured voice data SD and if applicable also classification of the object- and/or scene-related voice commands OSB, SSB contained therein based on statistical and/or artificial intelligence self-learning methods, particularly by using a neural network.

Besides the capture of image data BD in the course of the clinical studies conducted, a large number of voice commands SB passed to the camera guidance assistant by the surgeon for example are also captured, and characteristic voice features are determined therefrom using statistical and/or self-learning methods of artificial intelligence (“Deep Learning Methods”) particularly using deep neural networks, which features then form a “speech vocabulary” of a “self-learning” voice control routine SSR. The “self-learning” voice control routine SSR is also “trained” through evaluation of the large quantity of voice command sets obtained from the clinical studies, and voice command classes are formed therefrom. A “self-learning” voice control routine SSR of such kind is thus capable of capturing one or more phonetic sound sequence(s) together with breaks in speech and is then able to identify corresponding words or word combinations contained in the voice command SB or a voice command combination SBK from a captured phonetic sound sequence based on the trained neural network by applying word and/or syllable recognition algorithms. The detection and comparison with words or word combinations already stored in the “speech vocabulary” of the “self-learning” voice control routine SSR may be performed using a vector-based method, for example.

The object- and/or scene-related information OI, SI obtained by analysis of the image data BD are used to evaluate voice data SD with object- and/or site-related contents and are converted into corresponding control signals SS by the voice control routine SSR. An object-related voice command OSB is understood to be a word or word combination in the voice command SB or a voice command combination SBK which refer(s) to objects that are represented in image B. Similarly, scene-related voice commands SSB also relate to words or word combinations in a voice command SB or voice command combination SBK that relate to surgical scenes which are represented in image B and are also detected by the image analysis routine BAR, or derived surgical scenes, for example, the current orientation or position of one or more objects in image B.

The surgeon may thus engage in context-dependent and/or scene-dependent voice control using object- and/or scene-related voice commands OSB, SSB derived from the context of the current surgical scene, such as “Show the right surgical instrument” or “Show the scalpel” or “Show the gallbladder”.

Upon input of these object- and scene-related voice commands OSB, SSB, which are listed for exemplary purposes and comprise several words, the endoscope camera 20 for example is guided or dynamically guided by the surgery assistance system 1 in such manner that in image B the “surgical instrument” or the “scalpel” or the “gallbladder” is shifted from the right half of the image into the middle of the image, and optionally is also displayed larger or smaller. Thus, besides corresponding guidance of the endoscope camera 20, voice control of selected camera functions, such as “zoom”, or activation of special image filters is also possible.

However, the prerequisite for this is reliable object recognition and a corresponding assignment of the recognized or detected objects in image B through the determination of the associated coordinates X, Y of markers or marker points of the respective object in the screen coordinate system BKS. The knowledge gained about the surgical scene currently displayed in image B through the application of artificial intelligence methods is used according to the invention for context-dependent or site-dependent voice control of the surgery assistance system 1, in order to achieve user-friendly guidance of the endoscope camera 20 for the surgeon through the ability to use object-related and/or scene-related voice commands OSB, SSB during the surgical procedure, that is to say in real time.

For this purpose, the voice control routine SSR is configured to capture and evaluate complex voice commands SB and voice command combinations SBK, wherein a voice command combination SBK contains several words or several word components which may be formed by one or more voice commands or spoken comments SB, OSB, SSB. One word or several words may thus constitute a single voice command SB, OSB, SSB or part of a voice command SB, OSB, SSB, and a voice command combination SBK may also comprise several such voice commands SB, OSB, SSB.

When a voice command combination SBK comprising one or more words is input by the surgeon, the words of a voice command combination SBK must be input with gaps in speech yet immediately following one another and within a predetermined time interval. In this context, besides the object-related and/or scene-related information OI, SI according to the invention, the voice command combinations SBK may also contain control information such as direction information RI, speed information VI, and/or magnitude information BI. These control information items relate to control commands that are known per se relating to the movement of the endoscope camera 20 in the spatial coordinate system RKS. The voice commands SB or voice command combination SBK input by the surgeon is further processed in the form of voice data SD.

The voice data SD that are captured by the voice control routine SSR are evaluated based on the object- and/or scene-related information OI, SI which is either supplied currently by the image analysis routine BAR and/or has been stored in the memory unit MU previously as “knowledge base”. An object-related voice command OSB contained in the voice data SD relates to at least one item of the object-related item of information OI, wherein for the invention an item of object-related information OI is understood to mean an object or an item of information relating to the object represented in image B. A scene-related voice command SSB contained in the voice data SD is directed to an item of scene-related information, wherein an item of scene-related information is understood to be for example the positioning of one or more objects in image B or in the assigned image coordinate system BKS or the surgical scene as such terms assigned thereto, particularly technical terms.

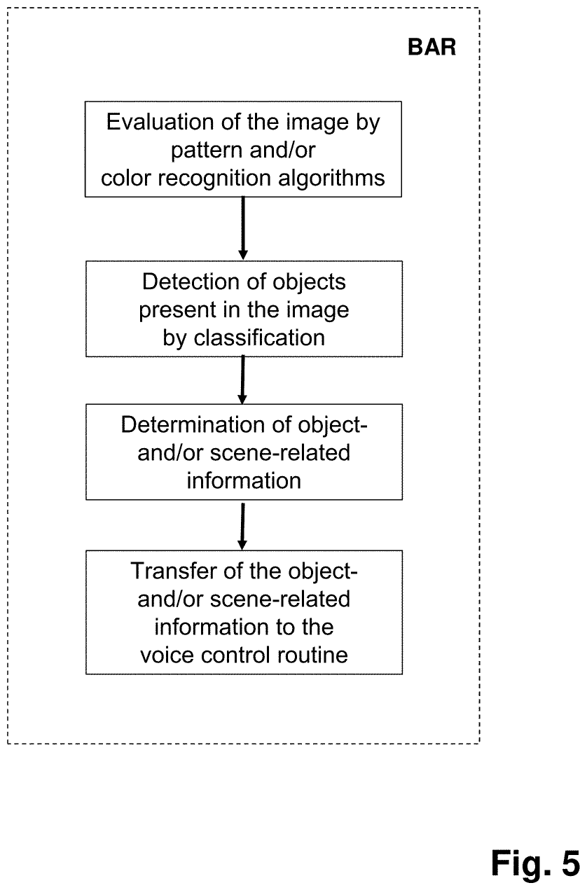

FIG. 5 shows an example of a schematic flowchart of the image analysis routine BAR according to the invention, which is executed in the control unit CU, which is preferably based on a trained neural network for evaluating the image data BD, via which the object- and/or scene-related information OI, SI used for context-dependent voice control is obtained.

When the image B currently being captured by the endoscope camera 20 has been made available in the form of image data BD, said data are evaluated by the image analysis routine BAR in the control unit CU, and the image data BD are analyzed and evaluated with the aid of the pattern- and/or color detection algorithms implemented in the image analysis routine BAR, and objects or parts thereof, particularly surgical instruments 30, 30a, 30b, or other medical tools or organs present in image B are detected. The detection is carried out based on a trained neural network that functions as the foundation for the image analysis routine BAR, using which markers or marker points such as an instrument tip S, Sa, Sb, particular color or material properties of the object or the articulation point between manipulator and instrument shaft of a surgical instrument 30, 30a, 30b are assigned to the individual objects, particularly the surgical instruments 30, 30a, 30b, by corresponding analysis of the image data BD.

A classification of the surgical scene and/or the objects located therein, i.e., a determination of object-related and/or scene-related information items OI, SI from the analyzed and evaluated image data BD, is then performed based on the markers or marker points detected in each case. In this process, preferably sequences and/or combinations of markers or marker points are checked via the neural network.

For example, the nature, the type, the properties, and/or the orientation of the objects detected in image B can be determined in the course of this process. An item of information obtained in this way relating to a classified object is then transferred either to the voice control routine SSR in the form of an object-related item of information OI for evaluation of incoming voice commands SB on the basis thereof. Additionally, the object-related item of information OI may also be stored in the memory unit MU.

An object-related item of information OI is the type of the surgical instrument 30, 30a, 30b displayed, for example, i.e., which surgical instrument 30, 30a, 30b specifically is displayed in image B, for example scissors or forceps. Additional characteristic markers or marker points for this specific surgical instrument 30, 30a, 30b may also be stored already in the trained neural network, via which further object-related information OI and/or scene-related information SI can be derived. For example, if the instrument tip S, Sa, Sb of a surgical instrument 30, 30a, 30b classified as scissors is defined as a marker or marker point, the orientation of the instrument tip S, Sa, Sb and therewith be inference the orientation of the surgical instrument 30, 30a, 30b classified as scissors in image B may also be calculated as scene-related information SI.

For this, it is necessary to define a unique assignment of the position of the markers or marker points of the detected surgical instrument 30, 30a, 30b and thus also the position of the surgical instrument in the screen coordinate system BKS. The position of individual marker or marker points of the detected surgical instrument 30, 30a, 30b is determined by calculating the corresponding coordinates Xa, Ya, Xb, Yb in the screen coordinate system BKS. The position of the detected object in image B, in the present case the detected surgical instrument 30, 30a, 30b, can be determined uniquely based on the associated coordinates Xa, Ya, Xb, Yb in the image coordinate system BKS depending on the detected object type and the associated markers or marker points. In FIG. 3, the coordinates Xa, Ya, Xb, Yb of the respective tip Sa, Sb of the first and second surgical instruments 30a, 30b are indicated in the screen coordinate system BKS for exemplary purposes.

Then, for example, it is possible to determine or calculate the distance Aa, Ab from the surgical instrument 30, 30a, 30b to the middle of the image B, that is to say to the origin of the screen coordinate system BKS, based on the coordinates Xa, Ya, Xb, Yb. For this, the distances Aa, Ab from predefined markers or marker points, such as from the instrument tip S, Sa, Sb to the middle of the image or the origin of the screen coordinate system BKS are preferably determined to obtain scene-related information SI about the orientation and/or position of the surgical instruments 30, 30a, 30b in image B. When a three-dimensional endoscope camera 20 is used, other additional coordinates (not shown in the figures) relating to depth information in a three-dimensional screen coordinate system comprising an additional z-axis may also be captured, based on which the distance between two objects in the operating space 12 can also be determined, which can then be used as a further open- or closed-loop control criterion.

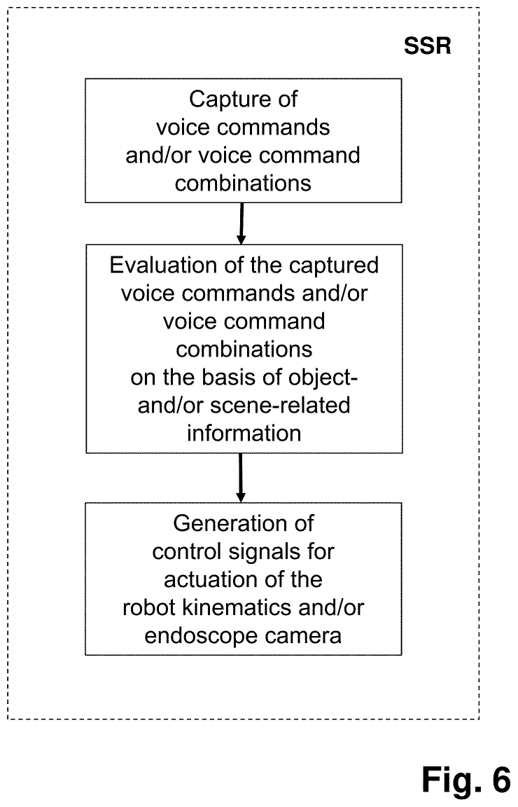

The object- and/or scene-related information items OI, SI provided by the image analysis routine BAR used in the voice control routine SSR to evaluate the captured voice commands or speech commands SB, particularly of object- and/or scene-related voice commands OSB, SSB. FIG. 6 shows an example of a schematic flowchart of an image analysis routine BAR according to the invention.

After the voice commands SB and/or voice command combinations SBK currently being input by the user or surgeon have been captured in the form of voice data SD, which may in particular also comprise object- and/or scene-related voice commands OSB, SSB, they are evaluated based on the supplied object- and/or scene-related information OI, SI. In this way, a direct temporal relationship is established between the image B which is currently displayed to the surgeon, the object- and/or scene-related information OI, SI derived therefrom, and the currently input object- and/or scene-related voice commands OSB, SSB. On this basis, the control signals SS are then generated for the corresponding actuation of the robot kinematics of the surgery assistance system 1 and/or the endoscope camera 20.

If the surgeon inputs the word combination “Show the right surgical instrument” as an example of a voice command combination SBK, the voice data SD obtained thereby are evaluated to determine whether there is a connection between the input words “Show”, “the”, “right”. “surgical instrument”, the order in which they are input, and/or the presence of speech gaps between them, and the calculated object- and/or scene-related information OI, SI. This is particularly advantageous possible when a correspondingly trained neural network is used which is part of the voice control routine SSR.

For this purpose, a comparison is made between the phonetic sound and/or syllable sequences contained in the voice data SD and the feature sequences or feature sequence combinations of the speech vocabulary stored in the voice control routine SSR and/or in the memory unit MU, and if individual feature sequences or feature sequence combinations match, an assigned object- and/or scene-related voice command OSB, SSB is detected and a control signal SS which is predefined or derived therefrom is generated. For example, besides the object-related information OI regarding the classified object “surgical instrument”, scene-related information SI in the form of the orientation of the classified object “surgical instrument” relative to the middle of image B may be stored, comprising position indicators such as “up”, “down”, “left” “right” as reference words, relative in each case to the middle of the image.

Accordingly, in the case of a surgical instrument positioned to the right of the image center in image B, the words “right” and “surgical instrument” are offered in the voice control routine SSR as an object- and/or scene-related voice commands OSB, SSB for evaluating the captured voice data SD in response to the provided object- and/or scene-related information OI, SI. A voice command SB is also assigned to the further voice command “Show” via the neural network, wherein the control command SS derived therefrom is dependent on the further object- and/or scene-related voice commands OSB, SSB “right” and “surgical instrument”. The voice control routine SSR also evaluates other scene-related information SI, which is supplied by the image analysis unit BAR, and also the coordinates Xb, Yb specified in the image coordinate system of the tip Sb of the second surgical instrument 30b represented as to the “right” of the image center and the calculated distance from the tip Sb to the image center, that is to say to the origin of the image coordinate system BKS. Alternatively, a vector V assigned to the second surgical instrument 30b may also be evaluated.

In response to the specified object- and/or scene-related information OI, SI, and the voice data SD captured by the input of the voice command “Show the right surgical instrument”, the voice control routine SSR generates the assigned control signals SS, based on which the endoscope camera 20 is moved by the surgery assistance system 1 in such manner that the tip Sb of the second surgical instrument 30b comes to rest in the middle of the image, that is to say on the origin of the image coordinate system BKS in image B of the endoscope camera 20.

The object- and/or scene-related information OI, SI provided continuously by the image analysis routine BAR is also made available continuously for processing of the captured voice commands SB by the voice control routine SSR.

The image analysis routine BAR and/or the voice control routine SSR may also be configured in such a manner that through the respective neural network the procedure history is considered in the evaluation of the image- and/or voice data BD, SD. For example, the current status of a minimally invasive surgical procedure may be captured by the image analysis routine BAR and/or the voice control routine SSR, and the endoscope camera 20 may be actuated such that the relevant “Field of Interest” is displayed automatically in the next operative step, i.e., without the input of any specific voice commands SB. For example, in the context of a gallbladder operation, an organ, an open artery which must be closed off in the next step, for example with a surgical clamp, may optionally already be enlarged in the display of image B.

In a design variant, voice control by the voice control routine SSR is only activated when an activation element, preferably an activation switch or button is operated by the surgeon or when a predefined voice activation command is uttered by the surgeon. This serves, in particular, to prevent voice commands SB from being input unintentionally by the surgeon.

The current coordinates X, Y of a reference point in image B or image coordinate system BKS may also be stored in a memory unit MU by corresponding voice input by the surgeon, for example, to make it easier to move to this reference point again later in the operation.

Besides capturing images via the endoscope camera 20, the blood circulation status of individual organs may also be captured, preferably using near-infrared techniques, and displayed in image B or captured an expanded image data with the image capture routine BER.

The invention was described in the preceding text with reference to exemplary embodiments thereof. Of course, many changes and modifications are possible without thereby departing from the inventive thought on which the invention is founded.

### LIST OF REFERENCE CHARACTERS

1 Surgery assistance system
2 Base unit
2.1 Carrier plate
2.2 Base housing
2.3 Fastening element
3 Support column
3′ Lower end section
3″ Upper end section
4 First robot arm
4′ First end section
4″ Second end section
5 Second robot arm
5′ First end section
5″ Second end section
6 Auxiliary instrument carrier
7 Angled joint part
8 Control device
10 Patient body
11 First surgical opening (“trocar”)
12 Operating space
13 Second surgical opening (“trocar”)
20 Endoscope camera
21 Endoscope
22 Camera unit
23 Microphone unit
30 Surgical instrument
30a First surgical instrument
30b Second surgical instrument
30′ Free end/hand grip region
30″ Free end
30a″ Free end of the first surgical instrument
30b″ Free end of the second surgical instrument
31 Hand grip elements
32 Function element(s)
Aa, Ab distance from origin
B Image
BAR Image analysis routine
BER Image capture routine
BD Image data
BI Magnitude information
BKS Image coordinate system
CU Control unit
LI Longitudinal axis
LK Longitudinal axis
OI Object-related information
OSB Object-related voice command
RI Direction information
RKS Spatial coordinate system
S Tip of the surgical instrument
Sa Tip of the first surgical instrument
Sb Tip of the second surgical instrument
SA1 First pivot axis
SA2 Second pivot axis
SA3 Third pivot axis
SA4 Fourth pivot axis
SA5 Fifth pivot axis
SB Control command
SBK Control command combination
SD Voice data
SI Scene-related information
SS Control signals
SSB Scene-related voice command
SSR Voice control routine
VI Speed information
x, y, z Spatial axes of the spatial coordinate system
X, Y Spatial axes of the image coordinate system
Xa, Ya Coordinates of an instrument tip
Xb, Yb Coordinates of an instrument tip
V Vector(s)
Z Further spatial axis of a three-dimensional image coordinate system

What is claimed is:

1. A surgery assistance system for guiding an endoscope camera, at least a section of the endoscope camera can be introduced through a first surgical opening and is movable in a controlled manner in an operating space of a patient body, the system comprising:
an endoscope camera for capturing images of the operating space in a form of image data, and
a robot kinematics, a free end of which accommodates the endoscope camera by an auxiliary instrument carrier, wherein the robot kinematics is movable in a motor-controlled manner for guiding the endoscope camera in the operating space, on a basis of control signals generated by a control unit, wherein at least one voice control routine is executed in the control unit, by which voice commands or voice command combinations in a form of voice data are captured, evaluated, and on the basis thereof the control signals generated by the control unit are generated, and at least one image capture routine being executed in the control unit for continuous acquisition of the image data relating to the operating space that are provided by the endoscope camera, wherein
at least one image analysis routine is provided in the control unit, by which the image data, previously captured, are continuously evaluated and classified on based upon statistical and/or artificial intelligence self-learning methods, that object and/or scene related information relating to a surgical scene currently being captured by the endoscope camera in an image is determined by the continuous evaluation and classification of the image data, and that the captured voice data are evaluated on the basis of the captured object and/or scene related information.

2. The surgery assistance system according to claim 1, wherein the image analysis routine comprises a neural network with pattern- and/or color detection algorithms for evaluating the captured image data.

3. The surgery assistance system according to claim 2, wherein the pattern and/or color detection algorithms are configured and trained to capture or detect objects or parts thereof which are present in the image, in surgical instruments, in medical tools, or in organs.

4. The surgery assistance system according to claim 1, wherein the voice control routine evaluates the voice data based on statistical and/or artificial intelligence self-learning methods.

5. The surgery assistance system according to claim 4, wherein the voice control routine comprises a neural network with sound and/or syllable recognition algorithms for evaluating the voice data.

6. The surgery assistance system according to claim 5, wherein the sound and/or syllable recognition algorithms are configured to capture sounds, syllables, words, gaps in speech and/or combinations thereof contained in the voice data.

7. The surgery assistance system according to claim 5, wherein the voice control routine is configured for an evaluation of the voice data on the basis of the object and/or scene related information.

8. The surgery assistance system according to claim 7, wherein the voice control routine captures object and/or scene related voice commands contained in the voice data, wherein at least one control signal is generated by the voice control routine on the basis of the object and/or scene related voice commands, previously captured, via which at least movement of the endoscope camera is controlled in terms of direction, speed and/or magnitude.

9. The surgery assistance system according to claim 4, wherein the voice control routine captures and evaluates directional and/or speed information and/or associated magnitude information in the voice data.

10. The surgery assistance system according to claim 1, wherein the endoscope camera is designed to capture a two-dimensional or three-dimensional image.

11. The surgery assistance system according to claim 1, wherein a two-dimensional image coordinate system or a three-dimensional image coordinate system is assigned to the image via the image analysis routine.

12. The surgery assistance system according to claim 11, wherein in order to determine an orientation and/or position of an object in the image coordinates (X, Y) of the object or at least of a marker or marker point of the object are determined in a screen coordinate system.

13. The surgery assistance system according to claim 1, wherein surgical instruments and/or organs and/or other medical tools displayed in the image are detected as objects or parts of objects by the image analysis routine.

14. The surgery assistance system according to claim 13, wherein in order to detect objects or parts of objects, one or more markers or marker points of an object is/are detected by the image analysis routine, wherein an instrument tip, special color or material properties of the object and/or an articulation point between a manipulator and an instrument shaft of a surgical instrument are used as markers or marker points.

15. The surgery assistance system according to claim 14, wherein the markers or marker points, previously detected, are evaluated by the image analysis routine for classifying the surgical scene and/or the objects located therein, and the object related and/or scene related information is determined on the basis thereof.

16. The surgery assistance system according to claim 15, wherein the object related and/or scene related information determined by the image analysis routine is transferred to the voice control routine.

17. A method for generating control signals for actuating robot kinematics movable in a motor-controlled manner of a surgery assistance system for guiding an endoscope camera comprising the steps of:
arranging the endoscope camera on a free end of the robot kinematics by an auxiliary instrument carrier;
introducing at least a section of the endoscope camera into an operating space of a patient body through a first surgical opening; and
executing at least one voice control routine in a control unit for generating the control signals, wherein by the voice control routine, voice commands and/or voice command combinations in a form of voice data are captured, evaluated, and the control signals are generated based thereon, and
executing at least one image capture routine in the control unit to continuously capture image data relating to the operating space supplied by the endoscope camera,
continuously classifying and evaluating the image data, previously captured, on based upon statistical and/or artificial intelligence self-learning methods by an image analysis routine executed in the control unit, that object and/or scene related information regarding a surgical scene currently captured in the image by the endoscope camera is calculated by the continuous evaluation and classification of the image data, and that the captured voice data are evaluated on the basis of the captured object and/or scene related information.

18. The method according to claim 17, wherein the captured image data are evaluated in the image analysis routine by pattern and/or color detection algorithms of a neural network.

19. The method according to claim 18, wherein the objects or parts of objects displayed in the image, surgical instruments or other medical tools or organs are detected by the pattern and/or color detection algorithms.

20. The method according to claim 19, wherein in order to detect the objects or parts of objects one or more markers or marker points of an object is/are detected by the image analysis routine, wherein an instrument tip, particular color or material properties of the object and/or an articulation point between a manipulator and an instrument shaft of a surgical instrument are used as markers or marker points.

21. The method according to claim 20, wherein the markers or marker points, previously detected, are evaluated by the image analysis routine in order to classify the surgical scene and/or the objects located therein, and object-related and/or scene-related information is determined on the basis thereof.

22. The method according to claim 21, wherein the object related and/or scene related information determined by the image analysis routine are transferred to the voice control routine.

23. The method according to claim 17, wherein the captured voice data are evaluated in the voice control routine by sound and/or syllable recognition algorithms of a neural network.

24. The method according to claim 17, wherein sounds, syllables, words, gaps in speech and/or combinations thereof contained in the voice data are captured by sound and/or syllable recognition algorithms.

25. The method according to claim 24, wherein object- and/or scene-related voice commands are captured by the voice control routine and are evaluated based upon transferred object- and/or scene-related information.

26. The method according to claim 17, wherein a two-dimensional image coordinate system is assigned to the image by the image analysis routine, and in order to determine orientation or position of an object in the image, coordinates (X, Y) of the object or at least one marker or one marker point of the object is/are determined in the screen coordinate system.
